(19) United States
(12) Patent Application Publication
(10) Pub. No.: US 2016/0256771 A1
(43) Pub. Date: Sep. 8, 2016

(54) SYSTEM FOR DIAGNOSTIC AND TREATMENT OF PHYSICAL AND COGNITIVE CAPABILITIES

(71) Applicant: INDIANA UNIVERSITY RESEARCH AND TECHNOLOGY CORPORATION, Indianapolis, IN (US)

(72) Inventors: Hamid R. Ekbia, Bloomington, IN (US); Pete Grogg, Bloomington, IN (US)

(73) Assignee: Indiana University Research and Technology Corporation, Indianapolis, IN (US)

(21) Appl. No.: 15/028,914

(22) PCT Filed: Oct. 9, 2014
(86) PCT No.: PCT/US2014/059811

§ 371 Date: Apr. 12, 2016

Related U.S. Application Data
(60) Provisional application No. 61/891,707, filed on Oct. 16, 2013.

(51) Int. Cl. A63F 13/20; A61B 5/16; A61B 5/11; A61B 5/00

(52) U.S. Cl. CPC A63F 13/20; A61B 5/4833; A61B 5/16; A61B 5/1128; A61B 5/744

(57) ABSTRACT

In one illustrative embodiment, a tele-rehabilitation platform allows patients to play and interact with therapists, caregivers, and other patients. The platform includes a set of computer games, a therapist portal, and a knowledgebase for the storage and analysis of therapy outcomes for specific patients, conditions, and therapy regimes.

### CROSS-REFERENCE TO RELATED APPLICATIONS

This application claims priority to U.S. Provisional Patent Application 61/891,707, filed Oct. 16, 2013, the entire disclosure of which is hereby expressly incorporated by reference.

### STATEMENT OF GOVERNMENTAL RIGHTS

This invention was made with government support under RR025761 awarded by the National Institutes of Health and 1314130 awarded by the National Science Foundation. The Government has certain rights in the invention.

### FIELD OF THE DISCLOSURE

This disclosure relates to physical rehabilitation. More specifically, this disclosure relates to an interactive tele-rehabilitation platform.

### BACKGROUND AND SUMMARY

With the rapid aging of the world's population, understanding how to promote health and wellbeing among older adults has become a public health priority. The aging of the population is creating a growing burden on medical professionals, families, and the government, all of whom are challenged with supplying the growing demand for healthcare of this population. Early detection of the onset of disease and early intervention are key to managing health in an optimal way.

Stroke is the number one cause of disability in the United States: 750,000 people experience a new or recurrent stroke annually. Of these, 400,000 (53%) survive with varying degrees of disability, resulting in tens of billions of dollars in health care costs (estimated at $70 billion in 2010). There are also about 600,000 Traumatic Brain Injuries (TBI's) a year. Spinal cord injuries (SCI) affect around another quarter of million Americans every year. Of these individuals, 89% are discharged from hospitals to a private home, and another 4.3% to nursing homes. Almost half of this population does not have insurance coverage.

Any improvement in the current therapeutic techniques would enhance the quality of life of tens of thousands of stroke, SCI and TBI survivors, and also translate into significant cost savings for the nation's health care system. Approximately 52% of SCI victims are considered paraplegic and 47% of quadriplegics suffer hemiparesis. It is the consequences of hemiparesis as well as the cardiac compromise that is a major factor in the reduced quality of life, i.e., there is reduced range of motion, strength, endurance, and balance/agility. In addition to these patient populations, a large group of geriatric and orthopedic patients can also benefit from the invention.

To date, multiple forms of technology-enabled interventions have been used with stroke and TBI patients: biofeedback, electrical stimulation, virtual and augmented reality, robotics, and video gaming. However, most of these alternative interventions require the use of expensive specialized equipment, including items directly worn on the body (e.g., headgear, cyber gloves, haptic technologies), making them prohibitively expensive and difficult to access and use.

When released, patients are expected to follow a prescribed sequence of therapeutic exercises primarily through individual initiative in the home or long-term care facility. This is supported by visits to a clinic where a therapist guides, evaluates performance, and adjusts the exercises as appropriate. The purpose of these exercises is to help the patient achieve functional goals (e.g., sitting and standing) as well as transitional goals (e.g., moving from sitting to standing). As noted, frequent repetition of each exercise is key to progression through the movement sequence and to achieving the functional goal.

These established rehabilitation methods, if followed by patients, have proven reasonably effective over time. However, success is limited by patient compliance with the prescribed exercises and by the ability of the therapist to monitor and guide the therapy. The therapist monitoring is limited to the clinic visits covered by insurance (typically only 10-20 if a person has insurance). Additionally, studies of compliance with prescribed exercises show that most patients will not or cannot persist on their own: only 31% of patients actually perform these exercises.

In a typical situation, healthcare providers prescribe home-based exercises (delivered on paper) for patients to perform on their own time. However, the rate of compliance of performing these home-based exercises is frequently low, and little if any data is captured or shared with the healthcare provider regarding exercise frequency, number of repetitions, quality of movement, etc. This may result in more frequent in-person meetings between the patient and healthcare provider, thereby potentially increasing expenses.

Multiple forms of tele-health interventions have been attempted to remotely assist patients wishing to improve or maintain physical functioning. Examples include video conferencing, tele-medicine gateways, home-based exercise instructions, and video gaming. These interventions are limited in their capabilities due to their inability to capture the full scope of functionality required to effectively guide, monitor, communicate, and track these patients and their progress.

The broad adoption of computing technologies in people's daily life has created the opportunity for ongoing interaction between patients and providers, as well as the accurate and objective monitoring of physical and cognitive activities. Currently, 79% of households in the U.S. own at least one computing platform (mobile, desktop, laptop, tablet), and 47% of them have internet connection at home. The key impediments in using this ubiquitous infrastructure for health-monitoring purposes are motivation, adherence, and privacy concerns. Video games have been shown to provide effective means for dealing with the motivation and adherence issues. In 2008, video games were played in 72% of American households, 23% of people 65+ played video games and 29% of all gamers were over the age of 50. Also, recent developments in cloud-based data communication protocols have largely alleviated technical issues related to privacy, although socio-psychological issues (e.g., the desire to conduct a dignified life) have yet to be dealt with. As part of this project, we will develop and evaluate a framework to deal with the issue of privacy.

The type of technology-enabled intervention addressed by the present disclosure includes platforms such as the Nintendo Wii™ and Microsoft Kinect™. However, some typical systems as currently available have limited functionality, require a kind dexterity not available to this patient population, and cannot be used for systematic data collection and analysis. Additional wearable monitoring technologies, such as Fitbit™ or Jawbone®, are also available.

A need therefore exists to address both issues of compliance and monitoring through technical and clinical innovations to allow for less-frequent in-person visits and to help the patient achieve short- and long-term functional/transitional goals. Such a need may addressed, for example, through a system in which physical and cognitive abilities can be monitored using in-home technologies with the results communicated to healthcare providers and caretakers in an ongoing basis using technology that is accessible and available in the home to develop a personal baseline of capabilities, which can further enable personalized treatment and intervention.

In one embodiment, an online platform featuring interactive technologies designed to achieve optimal outcomes for physical functioning is provided. The platform illustratively further addresses issues such as access, communication, compliance, monitoring, quality, care management, decision support, and the development of best practices. Motion detection/capture, gaming technologies, and location tracking technologies are used by the platform.

In one embodiment, a method of monitoring a patient is provided. The method includes prescribing a series of exercises for the patient to perform using a game interface; electronically monitoring the patient's performance of the series of prescribed exercises; and determining, based on said electronic monitoring, the patient's compliance with the prescribed series of exercises. In one more particular embodiment, the method further includes performing an assessment of the patient's physical functioning; determining goals for the patient; and determining a baseline functioning status of the patient and deficiencies based in part on the performed assessment of the patient's physical functioning and the determined goals for the patient; wherein the prescribed series of exercises are based on the determined baseline functioning status of the patient and deficiencies. In an even more particular embodiment, the method further includes performing a reassessment of the patient's physical functioning; and determining a progress status of the patient based on the performed reassessment. In yet an even more particular embodiment, the method further includes revising the determined goals for the patient based on the determined progress status; and prescribing a second series of exercises for the patient to perform using a game interface, the second series of exercises being based on the revised goals.

In another embodiment of any of the above methods, electronically monitoring the patient's performance includes monitoring the patient's performance of the series of prescribed exercises with a motion capture camera. In one more particular embodiment, electronically monitoring the patient's performance includes providing a time in which an exercise is to be completed and determining with the motion capture camera whether the patient performed the exercise in the provided time. In another more particular embodiment, electronically monitoring the patient's performance includes providing an exercise requiring at least one motion corresponding to at least one of a horizontal motion and a vertical motion, determining with the motion capture icon whether the patient has performed the motion. In still another more particular embodiment, the game interface includes at least one of a horizontal motion icon and a vertical motion icon, and displaying an avatar on the game interface indicating whether the patient has performed the motion. In yet still another more particular embodiment, electronically monitoring the patient's performance includes: displaying an avatar on the game interface; and moving the avatar on the game interface based on a movement of the patient as monitored by the motion capture camera. In an even more particular embodiment, electronically monitoring the patient's performance includes displaying a first target on the game interface, wherein the first target is positioned relative to the avatar such that a therapeutic movement must be performed by the patient to move at least a portion of the avatar to the first target. In an even more particular embodiment, electronically monitoring the patient's performance further includes displaying a second target on the game interface, wherein the second target is positioned relative to the first target such that a therapeutic movement must be performed by the patient to move at least a portion of the avatar from the first target to the second target.

In another more particular embodiment of any of the above embodiments, electronically monitoring the patient's performance includes monitoring the patient's performance of the series of prescribed exercises with an accelerometer, wherein the accelerometer is part of an activity monitor.

In another more particular embodiment of any of the above embodiments, the game interface includes a multi-player mode configured to allow the patient to engage with a second person. In an even more particular embodiment, the game interface is configured to adjust for a physical capability or skill of each player.

In one embodiment, a method of categorizing a patient's performance is provided. The method includes assessing the patient's physical functioning and determining at least one physical function dimension based on said assessment; assessing the patient's cognitive functioning and determining at least one cognitive function dimension based on said assessment; and assessing the patient's motivation and determining at least one motivation dimension based on said assessment; wherein at least one of said assessing steps include prescribing a task for the patient to perform and electronically monitoring the patient's performance of the task with a motion capture camera or accelerometer, wherein the accelerometer is part of an activity monitor. In one more particular embodiment, the at least one physical function dimension is selected from the group consisting of balance, coordination, range of motion, endurance, and strength. In another more particular embodiment, the at least one cognitive function dimension is selected from the group consisting of reaction time, free and cued recall, sensory/visual deficits, neglect, and comprehension. In still another more particular embodiment, the at least one motivation dimension is emotional arousal.

In one embodiment, as system for monitoring patient compliance is provided. The system includes a display configured to display information relating to a game or exercise for the patient to perform; a motion capture camera; a compliance module in communication with the display and the motion capture camera, wherein the compliance module is configured to receive one or more games or activities to be performed by the patient; wherein the compliance module is configured to determine whether a patient is performing the game or exercise based on a signal received from the motion capture camera; and wherein the compliance module is configured to record at least one of a number of repetitions attempted, a number of repetitions completed, a movement made by the patient, and an accuracy of a movement made by the patient. In a more particular embodiment, the system includes at least one accelerometer providing information related to a movement of the patient to the compliance module and the compliance module determines patient compliance at least in part based on the information provided by the accelerometer.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1A is a schematic of an exemplary embodiment of the online tele-health platform.

FIG. 1B is a schematic illustrative of a Game Interface module of the exemplary embodiment of FIG. 1A.

FIG. 1C is a schematic illustrative of a Middleware module of the exemplary embodiment of FIG. 1A.

FIG. 1D is a schematic illustrative of a Patient Management Dashboard module of the exemplary embodiment of FIG. 1A.

FIG. 2 is an illustrative view of a therapist login screen of the online tele-health platform of FIG. 1.

FIG. 3 is an illustrative view of a patient selection screen of the online tele-health platform of FIG. 1.

FIG. 4 is an illustrative view of an set-up screen of the online tele-health platform of FIG. 1.

FIG. 5 is an illustrative view of an alignment or calibration screen of the online tele-health platform of FIG. 1.

FIG. 6 is an illustrative view of an exemplary game view of the online tele-health platform of FIG. 1.

FIG. 7 is an illustrative view of a patient score screen of the online tele-health platform of FIG. 1.

FIG. 8A is an illustrative patient summary screen of the online tele-health platform of FIG. 1.

FIG. 8B is another illustrative patient summary screen of the online tele-health platform of FIG. 1.

FIG. 8C is still another illustrative patient summary screen of the online tele-health platform of FIG. 1.

FIG. 8D is still yet another illustrative patient summary screen of the online tele-health platform of FIG. 1.

FIG. 9 illustrates an exemplary method for use with a case management tool.

FIG. 10 illustrates an exemplary method for monitoring and recording patient compliance with a therapy regimen.

FIG. 11 is a schematic of an exemplary embodiment of the online tele-health platform including an accelerometer-based monitor.

FIG. 12 illustrates an exemplary method for categorizing a patient's performance trajectory.

FIG. 13 illustrates an exemplary method for tracking and reporting in-home monitoring dimensions.

Corresponding reference characters indicate corresponding parts throughout the several views. Although the drawings represent embodiments of the present invention, the drawings are not necessarily to scale and certain features may be exaggerated in order to better illustrate and explain the present invention. The exemplification set out herein illustrates an embodiment of the invention, in one form, and such exemplifications are not to be construed as limiting the scope of the invention in any manner.

### DETAILED DESCRIPTION

The embodiment disclosed below is not intended to be exhaustive or limit the invention to the precise form disclosed in the following detailed description. Rather, the embodiment is chosen and described so that others skilled in the art may utilize its teachings.

One of ordinary skill in the art will realize that the embodiments provided can be implemented in hardware, software, firmware, and/or a combination thereof. Programming code according to the embodiments can be implemented in any viable programming language such as C, C++, HTML, XTML, JAVA or any other viable high-level programming language, or a combination of a high-level programming language and a lower level programming language.

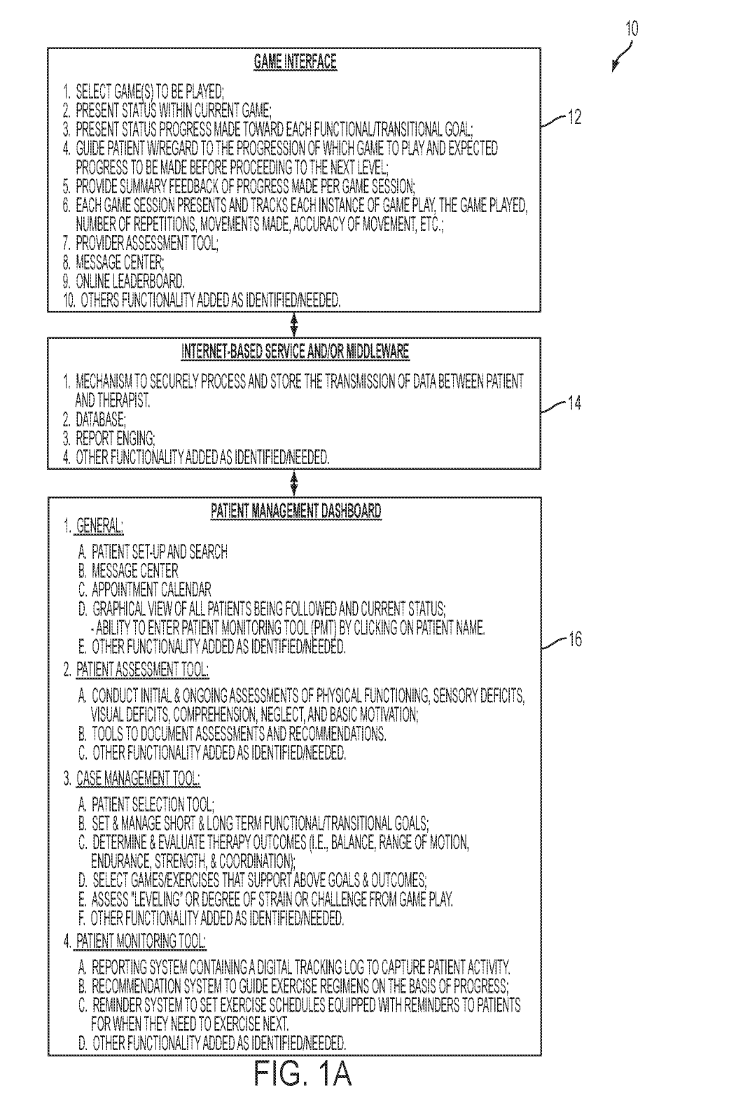

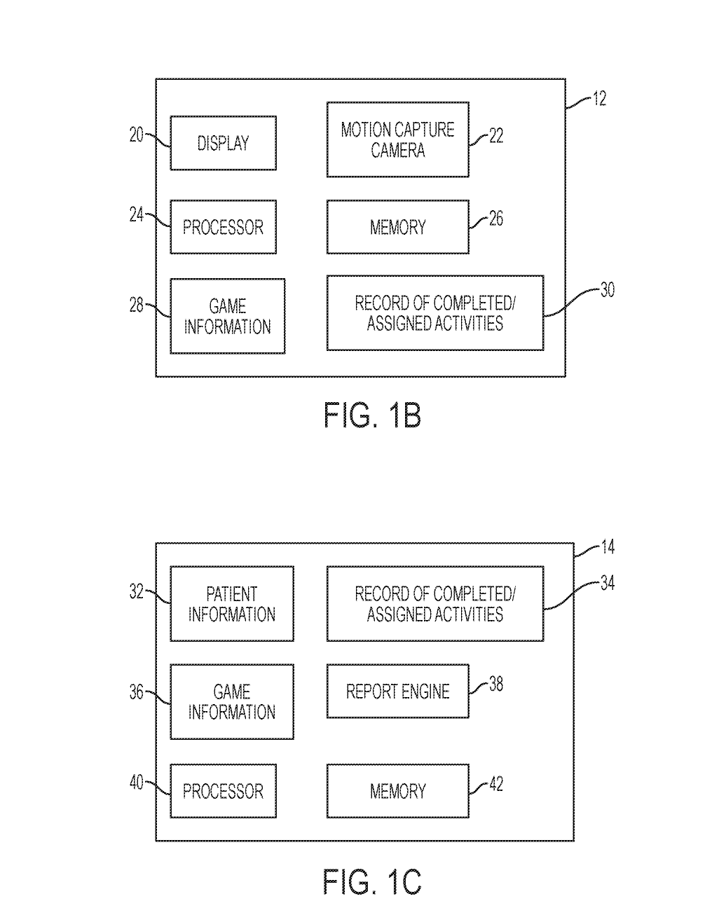

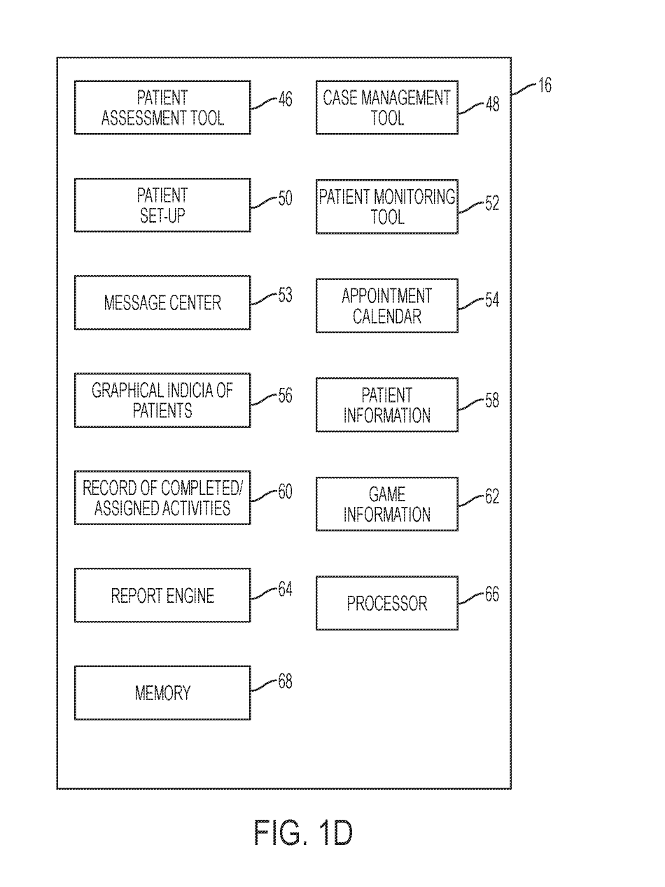

A schematic illustrating an exemplary tele-health system 10 is shown in FIG. 1. The system 10 is comprised of a plurality of user interfaces or modules. The system 10 includes a game interface 12 that is operatively connected to an internet-based service or middleware 14. The middleware 14 is operative connected to a patient management dashboard 16.

Using the patient management dashboard 16, healthcare providers select different games to be accessed by a patient through the game interface 12 that will help patients achieve specific functional/transitional goals. Exemplary patients include patients with brain injuries such as injuries caused by a stroke or TBI, and patients with burn injuries, orthopedic injuries, or another type of injury requiring physical therapy. An exemplary functional/transitional goal may be transferring from bed to a chair. To do this, a patient must be able to demonstrate balance, coordination, strength, and range of motion. The healthcare provider will select games using the patient management dashboard 16 under these categories that will then help the patient achieve this/these goal(s). The patient follows an exercise sequence with frequent repetition of each exercise utilizing the game interface 12 to achieving functional and transitional goals. As the patient works toward achieving these functional/transitional goals, the patient will be guided from game-to-game and within each game in the game interface 12 through specific therapeutic sequences (e.g., moving symmetrically from sitting to standing). The game interface 12 provides a system that can “observe”, measure, and evaluate specific motor movements as well as a game environment that will promote, guide, and provide feedback on the execution of those specific movements.

Referring to FIGS. 1A and 1B, the game interface module 12 may include one or more displays 20, such as a computer screen, television screen, or projector image. Game interface 12 further includes a motion capture camera 12. In some illustrative embodiments, existing game consoles and online gaming sites (such as Xbox Live or PlayStation Network) serve to facilitate the use of the platform by healthcare providers (i.e., physicians, therapists, physical trainers, etc.) and patients seeking to improve their physical functioning. For example, commercial available motion capture cameras, such as the Microsoft Kinect for the Xbox or windows, track the position of multiple skeletal points, such as 10, 15, 20, 25, 26, 30, or more skeletal points, to display an on-screen avatar of the patient. Game interface 12 may include logic controlling operating of game interface 12, which may be implemented in hardware or in hardware executing software. Exemplary software and databases may be stored in memory 26. Game interface 12 may include one or more processors 24 or other structures to implement the logic of middleware 14. As discussed in more detail below, game interface 12 may include or be in communication with an activity monitor 82, including an accelerometer-based activity monitor such a Fitbit™ or Jawbone® monitor (See FIG. 11). As indicated in FIG. 1A, game interface 12 may also provide other functionality as identified and/or needed.

Memory 26 is a non-transitory computer readable medium and may be a single storage device or may include multiple storage devices, located either locally with processor 24 or accessible across a network, or partially locally with processor 24 and partially accessible across a network. Computer-readable media may be any available media that may be accessed by processor 24 and includes both volatile and non-volatile media. Further, computer readable-media may be one or both of removable and non-removable media. By way of example, computer-readable media may include, but is not limited to, RAM, ROM, EEPROM, flash memory or other memory technology, CD-ROM, servers, Digital Versatile Disk (DVD) or other optical disk storage, magnetic cassettes, magnetic tape, magnetic disk storage or other magnetic storage devices, or any other medium which may be used to store the desired information and which may be accessed by processor 24. In one embodiment, processor 24 communicates data, status information, or a combination thereof to a remote device for storage, analysis, or carrying out a predetermined command. In another embodiment, memory 26 may further include operating system software. Memory 26 further includes communications software for communication with a network, such as a local area network, a public switched network, a CAN network, and any type of wired or wireless network. An exemplary public switched network is the Internet. Exemplary communications software includes e-mail software, SMS, Bluetooth communication software, radio frequency communication software, near field communication software and internet browser software. Other suitable software which permits processor 24 to communicate with other devices across a network may be used.

As illustrated in FIG. 1B, game interface 12 may include or have access to game information 28 and a record of completed or assigned activities 30. Within game interface 12, a user is first instructed to select the game to be played. The games incorporate the sequence of exercises and movements prescribed by a healthcare provider and intended to help patients achieve outcomes such as balance, coordination, range of motion, strength and endurance that support achieving the functional/transitional goals identified for each patient. Games are organized around these outcomes. The game interface 12 may display or present the current status of the user within the current game. The game interface 12 may further display or present the user's progress made towards one or more functional/transitional goals set by the patient, system 10, and/or healthcare provider.

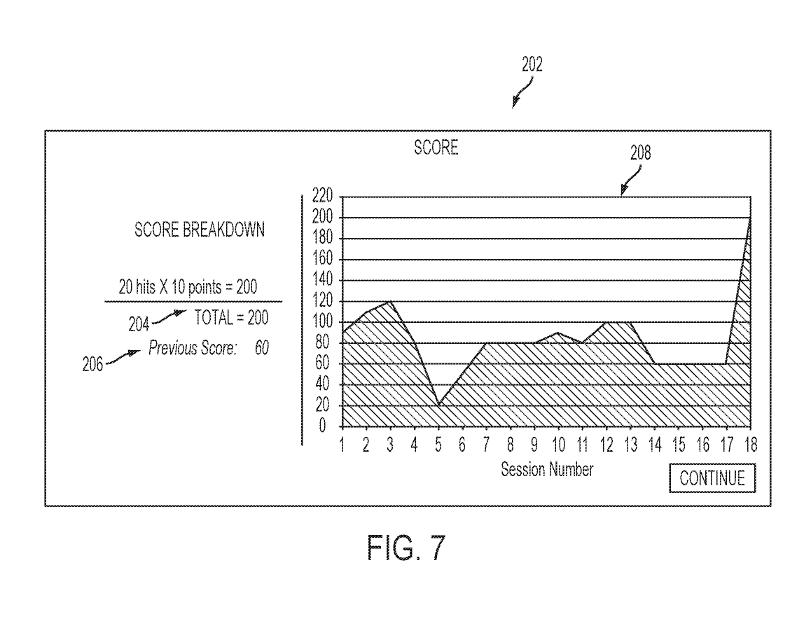

The game interface 12 further guides the user with regard to the progression of games to be played, and provides information relating to the expected progress to be made before proceeding to the next level or game. The game interface 12 may provide summary feedback of the progress made by the user in each game session. The game interface 12 may also provide information relating to each instance of game play, which game was played, the number of repetitions attempted and/or completed, the movements made, and/or the accuracy of those movements. Exemplary summary and session reports are further discussed with respect to FIGS. 7-11 below.

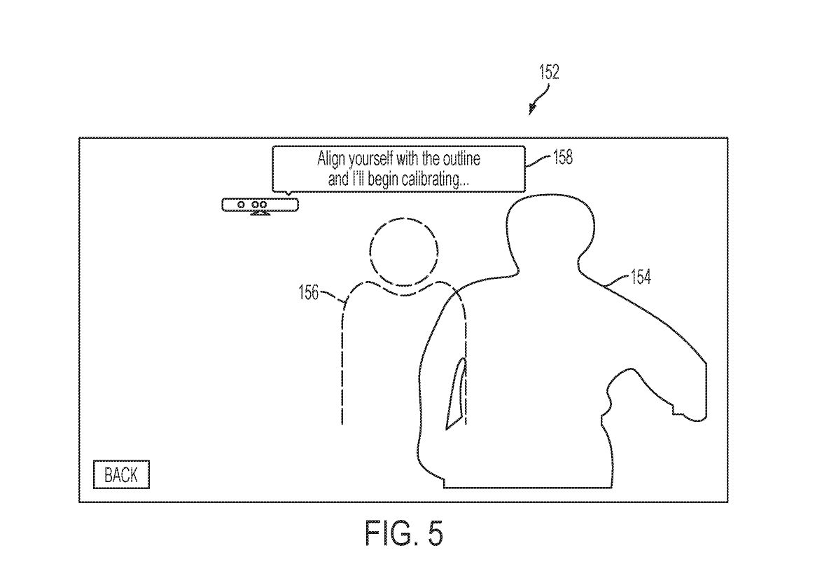

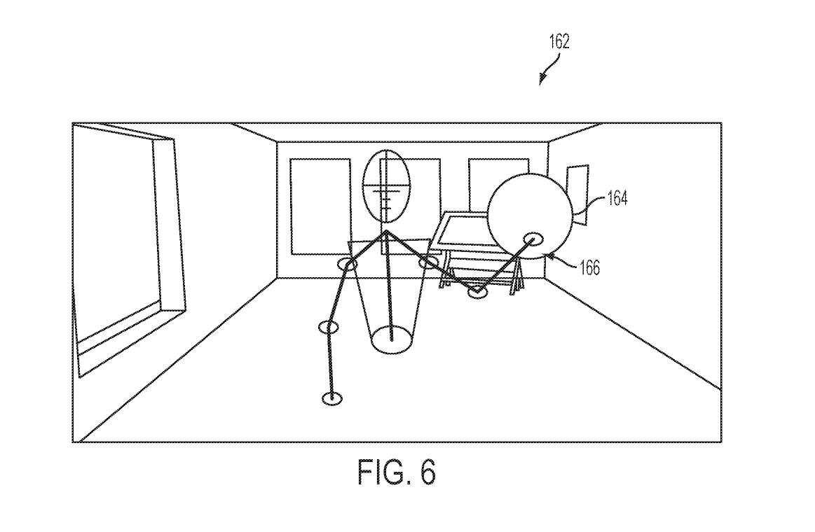

An exemplary game screen is shown in FIGS. 5 and 6. An alignment screen 152 is provided showing the position of an avatar 154 of the patient user as determined by the motion capture camera 22, as well as a desired user position 156. The user is directed by a prompt 158 to move his or her body until the position of the avatar 154 is aligned with the desired user position 156.

As shown in FIG. 6, the motion capture camera displays the current location of the user. The game screen 162 displays a target area 164 for the user. The exemplary game screen 162 illustrated in FIG. 6 is a basic game in which the user moves the avatar 154 to hit a stationary or falling ball or balloon at different angles and different distances from the avatar 154. To move the avatar, the user performs basic therapeutic movements of the upper extremities such as extension and scaption. The system 10 records performance metrics, such as, for example, response time measurements. In one embodiment, the system 10 monitors the progress of the patient user along a number of dimensions, allowing the health care professional to determine the level of exertion, and also the next steps in the therapy treatment, as well as the degree of compliance with the user patient's prescribed activities.

In one embodiment, the user is instructed to change position until the avatar 154 overlaps the target area 164. In some embodiments, the motion capture camera 22 tracks the location of one or more joints of the user, and displays these as part of the avatar 154. The game may instruct the user to maintain the position of the one or more joints 166 while moving another joint until the avatar 154 overlaps the target area 164.

As shown in FIG. 1, game interface 12 may also provide additional modules, such as a provider assessment tool, a message center, an online leaderboard comparing the user's results with those of other users, and other functionality as identified and/or needed.

A score report is shown in FIG. 7. In FIG. 7, a total score report 202 is shown. Illustratively, the report calculates a patient score 204 by multiplying the number of times a target was successfully achieved in a given time. The total score report 202 may show a previous score 206, or history of previous scores 208 in graphical form.

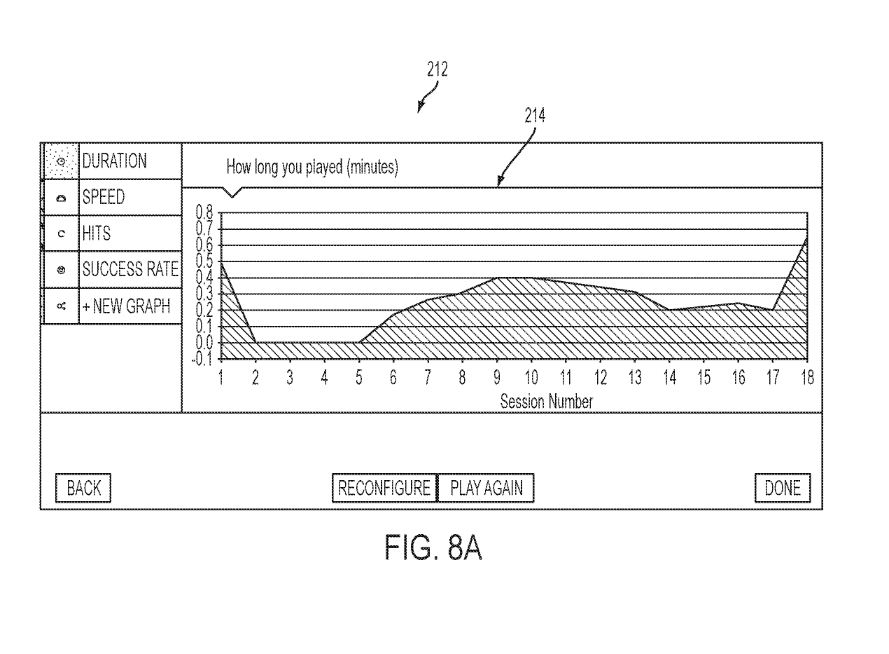

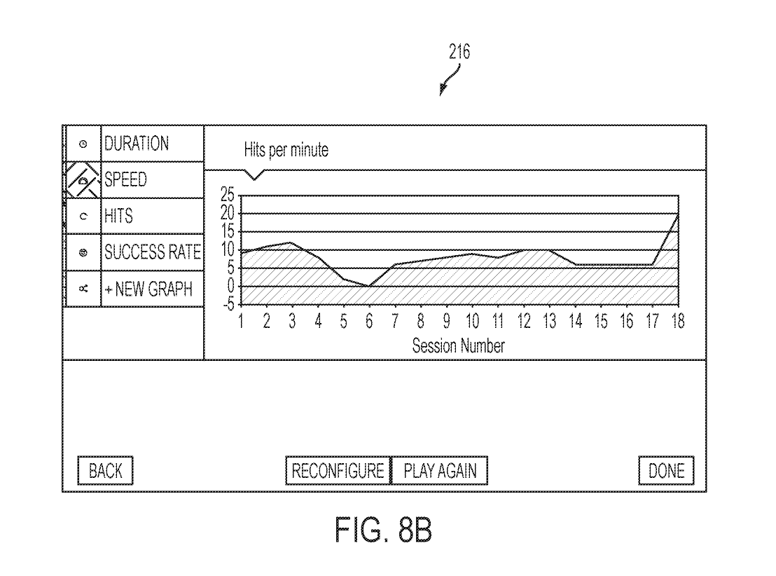

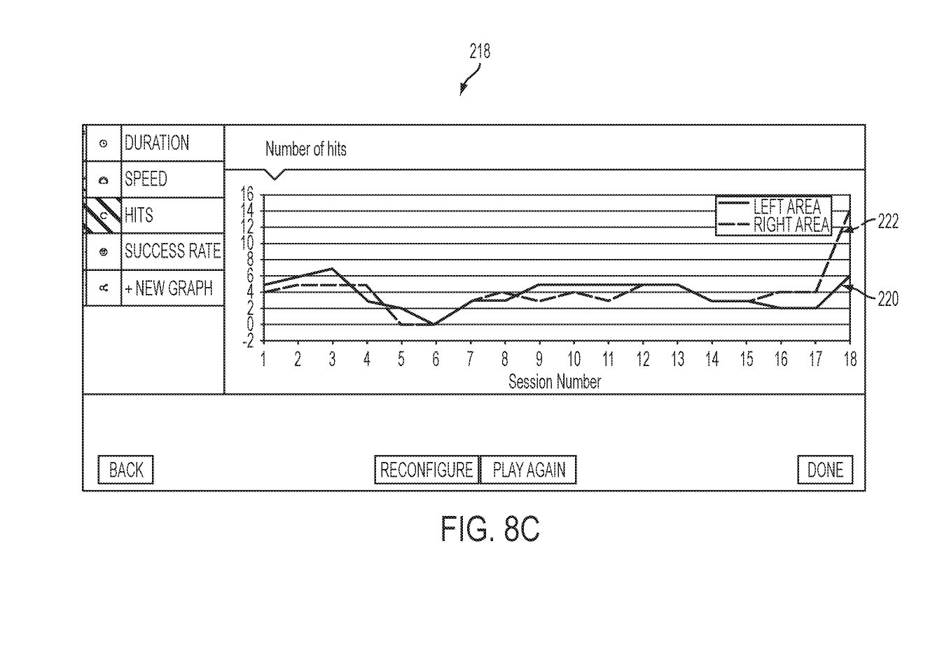

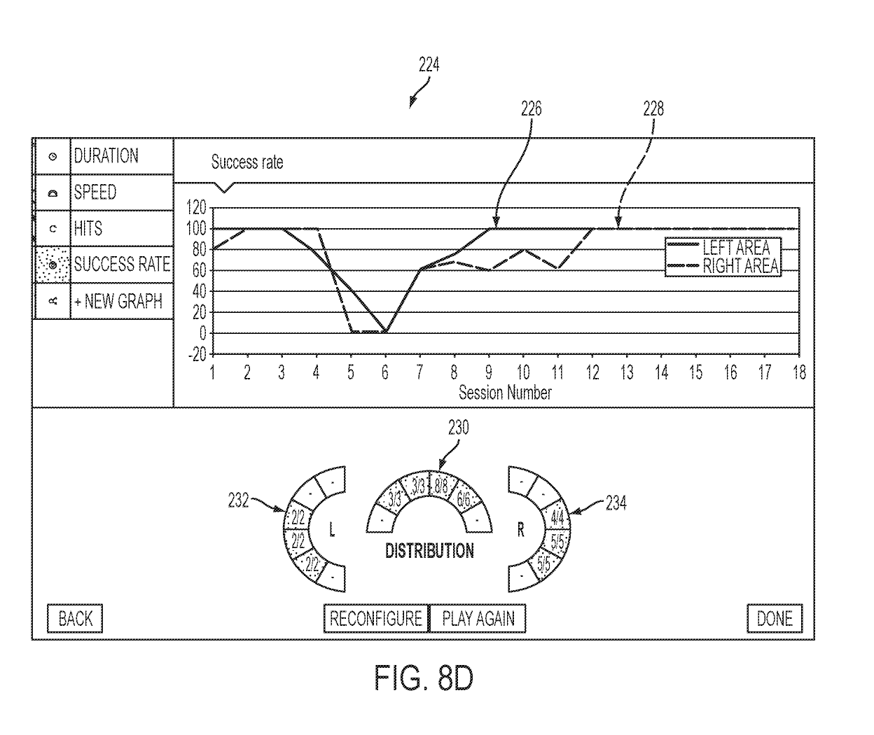

In FIGS. 8A-8D, a variety of summary screens are illustrated. In FIG. 8A, duration summary screen 212 is shown. The duration report 212 may show the time the previous exercises were played, and a comparison with the history of duration in graphical form 214. In FIG. 8B, a speed summary screen 216 is illustrated. In FIG. 8C, a hit summary screen 218 is illustrated. Illustrative hit summary screen 218 includes information for a user's left arm 220 and a user's right arm 222 on a single summary screen 218. In FIG. 8D, a success rate summary screen 224 is shown. Illustrative success rate summary screen 224 includes information for a user's left arm 226 and a user's right arm 228 on a single summary screen 224. Success rate summary screen 224 further includes a side distribution 230, showing the number of successes and attempts for each position indicated on the arcuate scale. Success rate summary screen 224 further includes a left side distribution 232, showing the number of successes and attempts for each horizontal position indicated on the arcuate scale, and a right side distribution 234, showing the number of successes and attempts for each horizontal position indicated on the arcuate scale.

Referring next to FIGS. 1A and 1C, the internet-based service and/or middleware 14 provides transmission and/or storage of data between the patient user and healthcare provider, such as a therapist. The middleware 14 is operatively connected to both the game interface 12 and the patient management dashboard 16. In another embodiment, game interface 12 is directly connected to the patient management dashboard 16. The internet-based service or middleware 14 may include or have access to patient information 32, and a record of completed and/or assigned games and activities 34. The results of one or more games or sessions engaged in by the user may be stored in one or more databases that are either part of or accessible by middleware 14. The middleware 14 may further include game information 36, and a report engine 38 for generating reports for the patient user or healthcare provider. Exemplary summary and session reports are further discussed with respect to FIGS. 7-11 below. Report engine 38 may generate other reports for the patient user, health care professional, healthcare provider, insurance carrier, or other entity. The one or more report engines may have access to the data stored in the one or more databases such that the data can be used to generate the reports. Report engine 38 may have access to the patient information 32, record of completed and/or assigned games and activities 34, and game information 36 of the middleware 14, or may have access to other databases, such as databases associated with the game interface 12 and the patient management dashboard 16. Middleware 14 may include logic controlling operation of middleware 14, which may be implemented in hardware or in hardware executing software. Exemplary software and databases may be stored in memory 42. Middleware 14 may include one or more processors 40 or other structures to implement the logic of middleware 14. As indicated in FIG. 1A, middleware 14 may also provide other functionality as identified and/or needed.

Referring next to FIGS. 1A and 1D, the system 10 further includes a patient management dashboard 16. In one embodiment, the patient management dashboard includes an Artificial Intelligence (AI) system to assist the healthcare provider in implementing and following a treatment plan, communicating with the patient.

The patient management dashboard 16 allows healthcare providers to review data captured for multiple patients during game play. The patient management dashboard 16 provides healthcare providers with a snapshot view of one or more patients being managed and the progress being made individually and collectively, allowing the healthcare providers to put patients in the relevant pools. The patient management dashboard 16 will also feature access to the patient assessment tool 46, the case management tool 48, patient set-up tool 50, and patient monitoring tool 52.

The patient management dashboard 16 includes general information for a healthcare provider, such as a physical therapist, to use and search using patient set-up tool 50, a message center 53, an appointment calendar 54, a graphical view 56 of one or more patients being monitored or followed by the healthcare provider that includes, for example, the patients' current status, and an ability to enter the patient monitoring tool by selecting that patient's name. As indicated in FIG. 1A, the patient management dashboard 16 may also include other functionality as identified/needed.

The level of physical functioning varies from one patient to the next in terms of symptoms, deficits, conditions, and capabilities. The variability derives from differences in the various physical, sensory, visual, or cognitive deficits as well as language comprehension, neglect, and motivation any patient may present. In one embodiment, the patient assessment tool (PAT) 46 provides a standardized tool allowing a healthcare provider to conduct initial and ongoing assessment of physical functioning from remote locations. The PAT 46 is used to conduct initial and ongoing assessments of physical functioning, sensory deficits, visual deficits, comprehension, neglect, and basic motivation. For initial assessment, PAT 46 will record baseline findings and identify deficiencies using an evidenced-based assessment tool and recommend a regimen or series of games to be followed. For ongoing, periodic assessment, PAT 46 will measure progress compared to initial baseline measures to allow the healthcare provider to adjust or create new short and/or long-term functional/transitional goals, document factors influencing progress.

Typically, a healthcare provider will visit with a patient at a location, such as a hospital, outpatient clinic, therapy center, or in the patient's home. Using the system 10, the frequency of in-person meetings may be decreased. Using the PAT 46, the healthcare provider will assess the patient and identify short and long-term functional/transitional goals. The healthcare provider will then design an exercise regimen with complimenting features offering both in-person visits and home-based exercises. Using a record of completed and/or assigned games and activities 60 captured and shared within the disclosure, a healthcare provider will determine when they should meet again in person.

The patient management dashboard 16 illustratively further includes a case management tool 48. The case management tool 48 provides the healthcare provider with the functionality to initiate and manage the care of each patient. In one embodiment, the case management tool 48 includes determining and evaluating therapy outcomes—e.g.; selecting games/exercises that support the above goals and outcomes; and assessing “leveling,” or the degree of strain or challenge on a certain exercise.

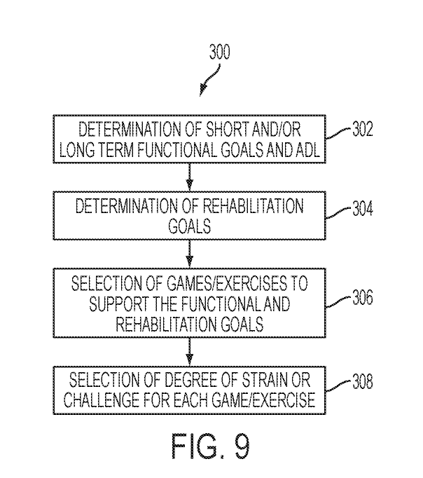

Referring next to FIG. 9, an exemplary method 300 for use with the case management tool 48 is illustrated. In step 302, the short and/or long term functional and transitional goals are identified. Exemplary functional goals and Activities of Daily Living (ADLs) include getting in and out of bed, and walking a certain distance. In step 304, the rehabilitation goals, or associated therapy outcomes associated with the functional goals, are identified. Exemplary rehabilitation goals include balance, range of motion, endurance, strength and coordination. Steps 302 and 304 may be completed by the health care professional in consultation with the patient. In step 306, the games and/or exercises supporting the functional and rehabilitation goals are identified. These games and exercises may be identified by the healthcare provider, or they may be automatically generated by the case management tool 48 based on the identified functional and/or rehabilitation goals. In step 308, the degree or level of strain or challenge for each game or exercise is determined. The degree or level may be determined by the healthcare provider, automatically generated by the case management tool 48, or both. The automatic generation in step 306 and/or 308 may also be based in part, on one or more of the following: the patient's medical history, the patient's past physical therapy compliance, and data from the current or past patients indicating successful strategies.

In one embodiment, the system 10 provides a series of games that incorporate a sequence of exercises and movements prescribed by a healthcare provider intended to achieve outcomes such as balance, coordination, range of motion, strength and endurance that support achieving the functional/transitional goals identified for each patient. This approach contrasts with efforts involving video games where the therapist must adapt therapy being delivered to games otherwise designed for entertainment.

In one embodiment, the system 10 captures data on compliance, progress, quality of movement, etc. and provides the data to the patient's healthcare provider to allow for remote monitoring and communication between or in lieu of in-person visits. The ability to demonstrate and monitor exercises is important to increased compliance. The use of gaming provides a strong motivational tool for the individual and promotes family participation and encouragement. Monitoring through a telemedicine interface permits the therapist to monitor data on compliance and success as well as to adjust the prescribed exercises without the need for an in-person visit. Remote monitoring will ensure higher compliance with home-based exercises. Patient progress with this platform provides the ability to have a greater timespan between in-person visits with the healthcare provider and, thus, minimize the number of times such visits must take place, providing added convenience and lower cost to the patient and healthcare provider.

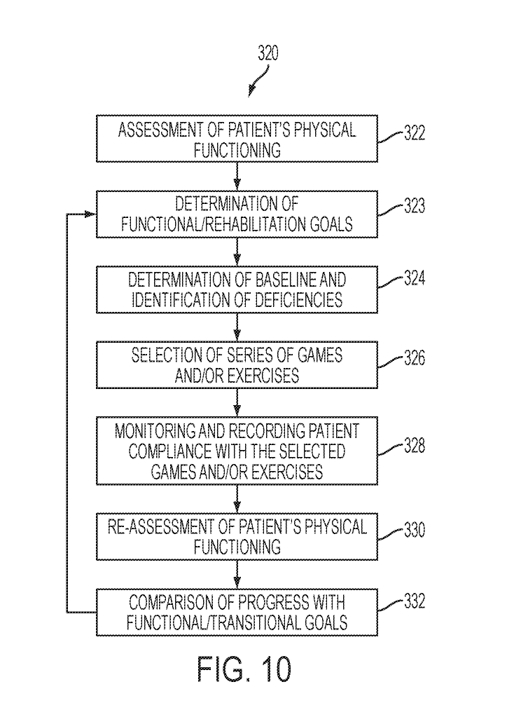

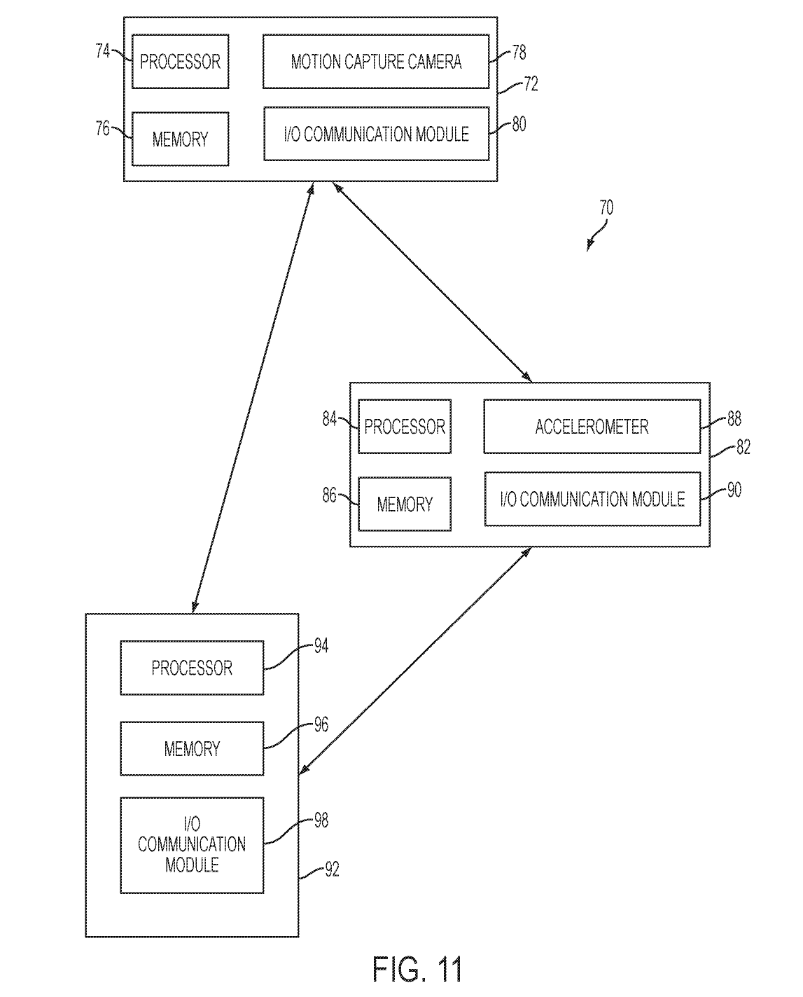

Referring next to FIG. 10 an exemplary method 320 for monitoring and recording patient compliance with a therapy regimen is illustrated. In step 322, an assessment of the patient's physical functioning is performed. Step 322 may be conducted using the PAT 46 described above. In step 323, functional and/or transitional goals for the patient are determined by the healthcare provider in consultation with the patient. Exemplary functional goals and Activities of Daily Living (ADLs) include getting in and out of bed, and walking a certain distance. In step 323, the rehabilitation goals, or associated therapy outcomes associated with the functional goals, are identified. Exemplary rehabilitation goals include balance, range of motion, endurance, strength and coordination. In step 324, the patient's assessment from step 322 is compared against the goals of step 323 to determine the patient's baseline functioning status and identify deficiencies. In step 326, games and/or exercises and difficulty level or degree supporting the functional and rehabilitation goals are identified. In step 328, the patient's compliance with the games and/or exercises determined in step 326 is monitored by the system 10, such as through the motion capture camera 22 of game interface 12.

Following a period of therapy, in step 330, the patient's physical functioning is reassessed, and in step 332, the patient's progress (based on the reassessment) towards the selected functional and rehabilitation goals is determined. The method 320 then returns to step 323 to determine whether the selected goals should be revised or are still applicable. A new baseline and deficiency identification is performed in step 324. The games and/or exercises in step 326 may be modified based on the new baseline and deficiencies of step 324. The patient's compliance with the new selected games and/or exercises is monitored and recorded in step 328.

The patient management dashboard 16 illustratively includes a patient monitoring tool 52. In one embodiment, the patient monitoring tool 52 includes a reporting system with a digital tracking log to capture patient activity and present it within the patient management dashboard 16; a recommendation system to guide exercise regimens on the basis of progress; and a reminder system to set exercise schedules equipped with reminders to patients of their next scheduled exercise session. In one embodiment, the system 10 may be used to connect healthcare providers and patient users separated by distance. The system 10 may be useful in multiple care settings, such as a hospital, long term care facility, rehabilitation hospital, outpatient clinic, or at home to improve access and delivery of therapy. Health conditions requiring exercise to improve or maintain one's physical functioning is another area of application. The knowledge resulting from this monitoring will also allow the therapist to adjust the timing of the limited number of covered clinical visits in order to maximize their value. The remote linkage will enable the delivery of reminders to patients in terms of the type and time of exercises and will permit the creation of a multi-player environment for socialization and competition (multi-player game play and an online leaderboard) between patients at other locations. For those without insurance, the discharging therapist can prescribe exercises that are then guided by the system 10, thus providing a low cost rehabilitation strategy.

In one embodiment, the system 10 provides automated reports to allow the healthcare provider to reduce manual documentation of therapy activities along with patient progress. Such documentation may be required by payers for both reimbursement purposes and as a basis for the continuation of therapy. Automated reports will reduce the time and effort needed for documentation through the automatic capture and analysis of data collected by the system 10.

The patient management dashboard 16 may further include or have access to additional patient information 58, and a record of completed and/or assigned games and activities 60 for each patient. The results of one or more games or sessions engaged in by the patient may be stored in one or more databases that are either part of or accessible by patient management dashboard 16. The patient management dashboard 16 may further include game information 62, and a report engine 64 for generating reports for the patient or healthcare provider. Patient management dashboard 16 may include logic controlling operation of patient management dashboard 16, which may be implemented in hardware or in hardware executing software. Exemplary software and databases may be stored in memory 68. Patient management dashboard 16 may include one or more processors 66 or other structures to implement the logic of patient management dashboard 16. As indicated in FIG. 1A, patient management dashboard 16 may also provide other functionality as identified and/or needed.

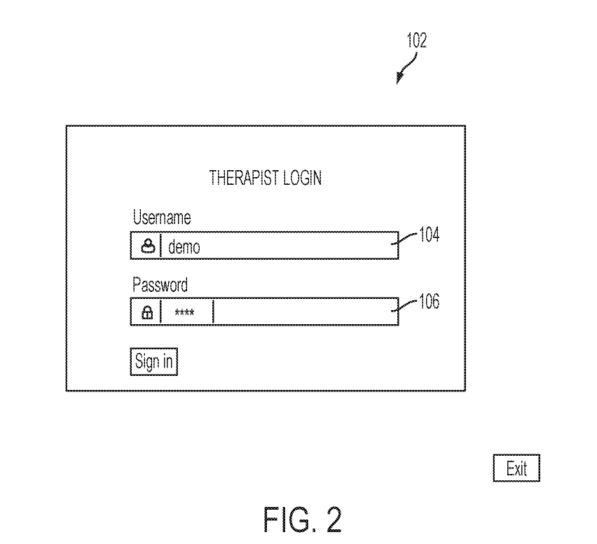

An exemplary log-on screen 102 for a healthcare provider is illustrated in FIG. 2. A healthcare provider, such as a therapist, enters a user name into box 104 and corresponding password into box 106. Upon determining a valid user name/password combination has been entered, the system 10 will provide the healthcare provider access to the system 10.

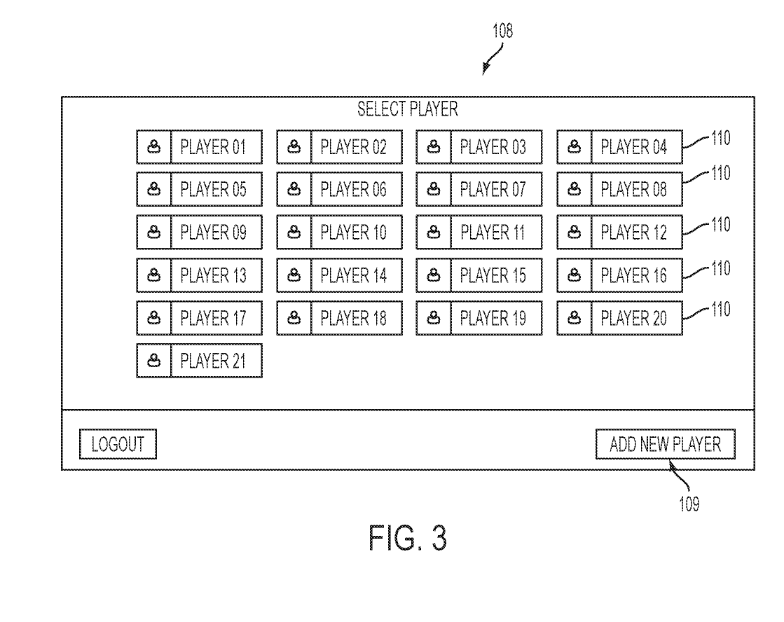

An exemplary patient selection screen 108 is shown in FIG. 3. One or more patients being tracked or monitored by the healthcare provider are each assigned their own icon 110. By selecting the icon 110 associated with a particular patient, the healthcare provider is provided access to information about that patient. Additional patients can be added by selecting the Add New Player icon 109.

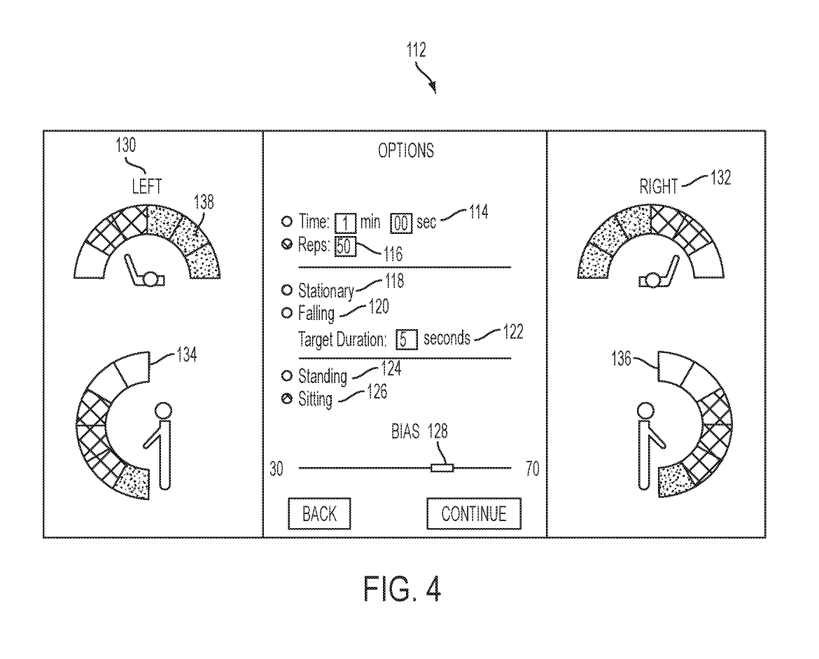

An exemplary game setup screen 112 is shown in FIG. 4. Game setup screen 112 includes exercise parameters, such as the time 114 to complete the assigned exercise or number of repetitions assigned 116. The healthcare provider can select whether the exercise is to be performed while stationary 118 or falling 120, and specify a target duration 122. The healthcare provider can further assign the activity to be performed while standing 124 or sitting 126. The healthcare provider can also set a bias 128 between the right and left side repetitions.

The game setup screen 112 further includes a left horizontal motion icon 130 and a right horizontal motion icon 132. The game setup screen 112 further includes a left vertical motion icon 134 and a right vertical motion icon 136. Each icon includes a scale, such as the arcuate scale shown 138, indicating an assigned range of motion. The healthcare provider can change the range of motion by selecting or deselecting the intervals in the arcuate scale. Other parameters, including time between repetitions, frequency of performing the exercise, and the like may also be set by the healthcare provider using the game setup screen 112.

### Group Tele-Health.

In one embodiment, a multi-player or rehab partner game play is provided. Patients may socialize, compete, and/or rehabilitate or train together within the tele-health platform. The multi-play games enhance engagement with the games and provide a social networking experience, further motivating and engaging patients by allowing them to support or compete with one another while playing with family, friends, or other patients either locally or online.

### Handicapped Multi-Player Game Play

In one embodiment, game play between multiple players is adjusted by the system 10 to adjust for different physical capabilities and/or skills of each player. However, to accomplish this, game play or competition will be “leveled” to allow for fair competition between two or more individuals possessing different physical capabilities/skills (i.e., healthy grandson vs. stroke survivor). The system 10 will provide a series of in-game body tests when setting up the game play persona or avatar to allow the system 10 to learn the player's physical abilities. The system 10 will then adjust or level the range of motions or degree of difficulty for the game for each player based on each player's physical abilities, allowing for a comparable level of difficulty for each player based on their individual abilities. This provides a healthy player the ability to have a level competition with a patient undergoing physical therapy or otherwise having a different skill level. As the patient's physical functioning improves, it will have a corresponding effect to the “healthy players” handicap. The handicapped multi-player game play provides an experience to help educate the family/caregiver(s) of the patient on what the patient is experiencing from a physical perspective and possibly from a cognitive perspective.

### Development of Best Practices and Decision Support.

In one embodiment, the system 10 includes the ability to track progress against identified deficiencies, short and long-term goals and expected therapy outcomes. Data collected across multiple patients, patient types, exercises, techniques, movements, etc. can be used to identify those games and corresponding exercises or combination of games achieving the best outcome(s). The gathered data is collected by the PAT 46 and patient monitoring tool 52 and the system 10 will recommend games or combination of games to a new patient that have been demonstrated in previous patients to achieve desired outcomes. Reports generated by this data may also be useful for research and quality improvement activities at user institutions.

Secure Tele-Health within a Game Environment.

In a typical current system, online gamers have accounts where personal information such as name, email, game statistics, and credit card information is stored for both online game play and online purchases within sites such as Xbox Live or PlayStation Network. These accounts may not meet the health industry standards or be in compliance with the health security requirements of the HITECH Act and HIPPA. In one embodiment, the system 10 includes security protocols to enable the secure transmission and storage of personal health information via online gaming sites.

### Accelerometer-Based Activity Monitor

Referring next to FIG. 11, a schematic of another exemplary embodiment of an online tele-health platform 70 is illustrated. Platform 70 illustratively includes gaming platform 72. Gaming platform 72 may be or may include game interface 12 as described in more detail above. Gaming platform 72 illustratively includes a processor 74 implementing logic controlling operation of the gaming platform 72, which may include hardware or in hardware executing software. Exemplary software and databases may be stored in local memory 76, or may be stored in a remote location, such as memory 86 or memory 98, that gaming platform 72 has access to. Gaming platform 72 further includes a motion capture camera 78, such as motion capture camera 12 described above. Exemplary commercially available motion capture cameras include the Microsoft Kinect™ for the Xbox or Windows. Gaming platform further includes a communication module 80 for communicating with activity monitor 82 and/or remote device 92. In some embodiments, communication module 80 communicates with communication module 90 of activity monitor 82 and/or communication module 98 of remote device 92 over a network such as a local area network, a public switched network, a CAN network, and any type of wired or wireless network. An exemplary public switched network is the Internet. In other embodiments, communication module 80 communicates directly with communication module 90 of activity monitor 82 and/or communication module 98 of remote device 92 over a wired or wireless connection.

Platform 70 further includes an activity monitor 82. Exemplary commercially available activity monitors 70 include Fitbit™ and Jawbone®. Other exemplary activity monitors include smart phones, tablets, and other computing devices. In some embodiments, activity monitor 82 includes a processor 84 implementing logic controlling operation of the gaming platform 82, which may include hardware or in hardware executing software. Exemplary software and databases may be stored in local memory 86, or may be stored in a remote location, such as memory 76 or memory 98, that activity monitor 82 has access to. Activity monitor 82 illustratively includes an accelerometer. In some embodiments, communication module 90 communicates with communication module 80 of gaming platform 72 and/or communication module 98 of remote device 92 over a network as described above, a wired connection, or a wireless connection.

Platform 70 further includes remote device 92. Exemplary remote devices 92 include desktop computers, laptop computers, tablet computers, or servers. In some embodiments, remote device 92 is or is part of system 10 implementing interface 12, middleware 14, and/or dashboard 16 as described above. In some embodiments, remote device 92 includes a processor 96 implementing logic controlling operation of the remote device 92, which may include hardware or in hardware executing software. Exemplary software and databases may be stored in local memory 98, or may be stored in a remote location, such as memory 76 or memory 86, that remote device has access to. Remote device communicates with communication module 80 of gaming platform 72 and/or communication module 90 of activity monitor over a network as described above, a wired connection, or a wireless connection.

Patient Evaluation with the Patient Assessment Tool (PAT)

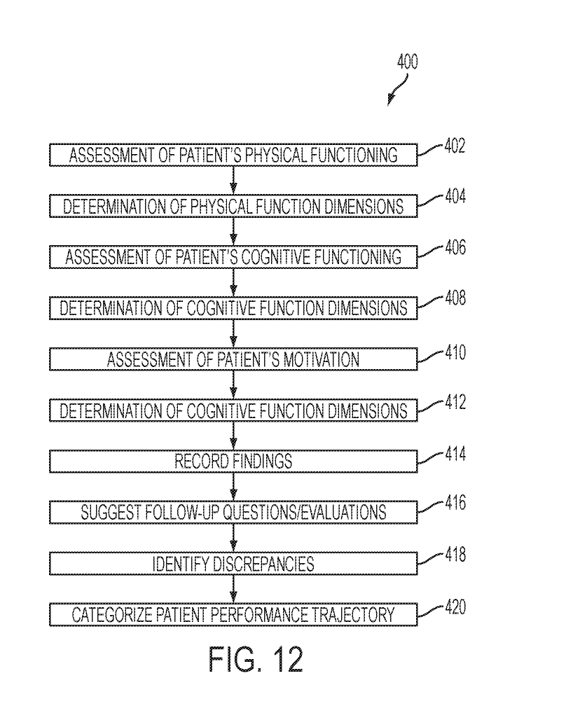

Referring next to FIG. 12, an exemplary method 400 for categorizing a patient's performance trajectory is provided. Although illustrated in a particular order the steps of method 400 are not intended to be limited to the order shown. For example, in some embodiments, blocks 402, 406, and 410 may be performed in any order before blocks 404, 408, and 412. In other embodiments, blocks 406 and 408 may be performed before block 404 and/or block 406, and blocks 410 and 412 may be performed before any of blocks 402-408. In some embodiments, method 400 is performed by PAT 46 (see FIG. 1D).

In block 402, the PAT 46 assess the patient's physical functioning. In some embodiments, the patient performs a series of games and/or exercises assigned by the PAT 46. Data from motion capture camera 78 and or accelerometer 88 related to these games and/or exercises is provided to PAT 46. In block 404, PAT 46 determines one or more physical functioning dimensions. Exemplary physical functioning dimensions include balance, coordination, range of motion, endurance, and strength.

In block 406, the PAT 46 assess the patient's cognitive functioning. In some embodiments, the patient performs a series of games and/or exercises assigned by the PAT 46. These games and/or exercises may be the same or different than the games and/or exercises assigned to assess the patient's physical functioning dimensions above. Data from motion capture camera 78 and or accelerometer 88 related to these games and/or exercises is provided to PAT 46. In block 408, PAT 46 determines one or more cognitive functioning dimensions. Exemplary cognitive functioning dimensions include reaction time, free and cued recall, sensory/visual deficits, neglect, and comprehension.

In block 410, the PAT 46 assess the patient's motivation. In some embodiments, the patient performs a series of games and/or exercises assigned by the PAT 46. These games and/or exercises may be the same or different than the games and/or exercises assigned to assess the patient's physical functioning and cognitive functioning dimensions above. Data from motion capture camera 78 and or accelerometer 88 related to these games and/or exercises is provided to PAT 46. In block 412, PAT 46 determines one or more motivation dimensions. An exemplary motivation dimension is emotional arousal.

In block 214, PAT 46 records the dimensions determined in blocks 404, 408, and 412. Based on the determined dimensions, PAT 46 in block 416 may suggest follow-up questions and/or evaluations to be conducted by the patient and/or the patient's health care provider. In block 418, PAT 46 identifies an discrepancies in the recorded dimensions. In block 420, PAT 46 proposes a patient category in terms of the trajectory of performance. The proposed category in block 420 may be based at least in part on the dimensions determined in blocks 404, 408, and 412, including any changes from one or more previous determinations of the dimensions. In some embodiments, method 400 is performed by PAT 46 on a regular basis, such as weekly, to monitor and update the current status of the patient's performance trajectory.

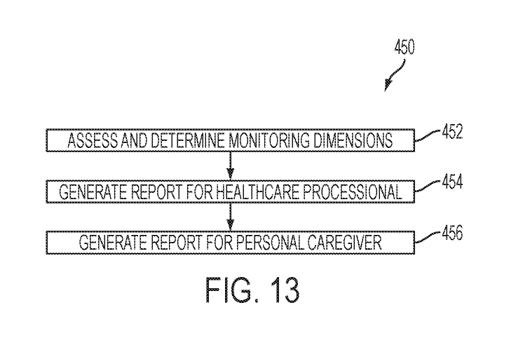

Referring next to FIG. 13, and exemplary method 450 for tracking and reporting in-home monitoring dimensions. In some embodiments, method 450 is at least partially performed by report engine 64 of patient management dashboard 16 (see FIG. 1D).

As shown in block 452 of FIG. 13, one or more monitoring dimensions are assessed and determined. In some embodiments, block 452 is performed by PAT 46, such as by method 400. Exemplary monitoring dimensions include physical functioning dimensions such as balance, coordination, range of motion, endurance, and strength, cognitive functioning dimensions such as reaction time, free and cued recall, sensory/visual deficits, neglect, and comprehension, and motivation dimension such as emotional arousal.

In block 454, report engine 64 generates a report for a healthcare professional at least partially based on the one or more monitoring dimensions determined in block 456. In block 456, report engine 64 generates a report for a personal caregiver at least partially based on the one or more monitoring dimensions determined in block 456. The reports generated in blocks 454 and/or 456 may be further based on additional data, such as the record of completed and/or assigned activities 60 (see FIG. 1D). Although FIG. 13 illustrates both a report for a healthcare professional being generated in block 454 and a report for a personal caregiver being generated in block 456, in some embodiments, only one report may be generated. In other embodiments, the reports may be generated simultaneously, or sequentially in any order.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A method of monitoring a patient comprising:
providing a patient monitoring system comprising an electronic game interface module, a patient management dashboard module, and a patient assessment tool module;
prescribing a series of exercises with the patient management dashboard module for the patient to perform using a game interface;
electronically monitoring the patient's performance of the series of prescribed exercises with at least one of a motion capture camera and an accelerometer; and
determining with the patient assessment tool module, based on said electronic monitoring, the patient's compliance with the prescribed series of exercises.

2. The method of claim 1, further comprising:
performing an assessment of the patient's physical functioning;
determining goals for the patient; and
determining a baseline functioning status of the patient and deficiencies based in part on the performed assessment of the patient's physical functioning and the determined goals for the patient;
wherein the prescribed series of exercises are based on the determined baseline functioning status of the patient and deficiencies.

3. The method of claim 2, further comprising:
performing a reassessment of the patient's physical functioning;
determining a progress status of the patient based on the performed reassessment.

4. The method of claim 3, further comprising:
revising the determined goals for the patient based on the determined progress status; and
prescribing a second series of exercises for the patient to perform using a game interface, the second series of exercises being based on the revised goals.

5. The method of claim 1, wherein electronically monitoring the patient's performance includes monitoring the patient's performance of the series of prescribed exercises with a motion capture camera.

6. The method of claim 5, wherein electronically monitoring the patient's performance includes providing a time in which an exercise is to be completed and determining with the motion capture camera whether the patient performed the exercise in the provided time.

7. The method of claim 5, wherein electronically monitoring the patient's performance includes providing an exercise requiring at least one motion corresponding to at least one of a horizontal motion and a vertical motion, determining with the motion capture camera whether the patient has performed the motion.

8. The method of claim 7, further comprising displaying an avatar on the game interface indicating whether the patient has performed the motion, wherein the game interface includes at least one of a horizontal motion icon and a vertical motion icon.

9. The method of claim 5, wherein electronically monitoring the patient's performance includes:
displaying an avatar on the game interface; and
moving the avatar on the game interface based on a movement of the patient as monitored by the motion capture camera.

10. The method of claim 9, wherein electronically monitoring the patient's performance includes displaying a first target on the game interface, wherein the first target is positioned relative to the avatar such that a therapeutic movement must be performed by the patient to move at least a portion of the avatar to the first target.

11. The method of claim 10, wherein electronically monitoring the patient's performance further includes displaying a second target on the game interface, wherein the second target is positioned relative to the first target such that a therapeutic movement must be performed by the patient to move at least a portion of the avatar from the first target to the second target.

12. The method of claim 1, wherein electronically monitoring the patient's performance includes monitoring the patient's performance of the series of prescribed exercises with an accelerometer, wherein the accelerometer is part of an activity monitor.

13. The method of claim 1, wherein the game interface includes a multi-player mode configured to allow the patient to engage with a second person.

14. The method of claim 13, wherein the game interface is configured to adjust for a physical capability or skill of each player.

15. A method of categorizing a patient's performance comprising:
providing a patient monitoring system comprising an electronic game interface module, a patient management dashboard module, and a patient assessment tool module;
assessing the patient's physical functioning using the electronic game interface module and determining with the patient assessment tool module at least one physical function dimension based on said assessment;
assessing the patient's cognitive functioning using the electronic game interface module and determining with the patient assessment tool module at least one cognitive function dimension based on said assessment; and
assessing the patient's motivation using the electronic game interface module and determining with the patient assessment tool module at least one motivation dimension based on said assessment;
wherein at least one of said assessing steps include prescribing a task for the patient to perform and electronically monitoring the patient's performance of the task with a motion capture camera or accelerometer, wherein the accelerometer is part of an activity monitor.

16. The method of claim 15, wherein the at least one physical function dimension is selected from the group consisting of balance, coordination, range of motion, endurance, and strength.

17. The method of claim 15, wherein the at least one cognitive function dimension is selected from the group consisting of reaction time, free and cued recall, sensory/visual deficits, neglect, and comprehension.

18. The method of claim 15, wherein the at least one motivation dimension is emotional arousal.

19. A system for monitoring patient compliance comprising:
a display configured to display information relating to a game or exercise for the patient to perform;
a motion capture camera;
a compliance module in communication with the display and the motion capture camera, wherein the compliance module is configured to receive one or more games or activities to be performed by the patient; wherein the compliance module is configured to determine whether a patient is performing the game or exercise based on a signal received from the motion capture camera; and wherein the compliance module is configured to record at least one of a number of repetitions attempted, a number of repetitions completed, a movement made by the patient, and an accuracy of a movement made by the patient.

20. The system of claim 19, further comprising at least one accelerometer, and wherein the accelerometer provides information related to a movement of the patient to the compliance module and the compliance module determines patient compliance at least in part based on the information provided by the accelerometer.
